(19) United States
(12) Patent Application Publication
(10) Pub. No.: US 2026/0287542 A1
(43) Pub. Date: Sep. 24, 2026

(54) ELECTROCHEMICAL APTAMER BIOSENSOR FOR DETECTING PHENYLALANINE AND/OR TYROSINE

(71) Applicant: Seoul Women's University Industry-University Cooperation Foundation, Seoul (KR)

(72) Inventors: Chang Seuk LEE, Gimpo-si, Gyeonggi-do (KR); JAEMIN JEONG, Uijeongbu-si, Gyeonggi-do (KR); INSEON YU, Seoul (KR)

(21) Appl. No.: 19/307,418

(22) Filed: Aug. 22, 2025

(30) Foreign Application Priority Data
Feb. 6, 2025 (KR) 10-2025-0015349

(51) Int. Cl. G01N 27/327; G01N 33/53; G01N 33/543; G01N 33/68

(52) U.S. Cl. CPC G01N 27/3276; G01N 33/5308; G01N 33/5438; G01N 33/6812; G01N 2800/04

(57) ABSTRACT

The present disclosure relates to an electrochemical aptamer biosensor capable of rapidly and easily detecting phenylalanine and/or tyrosine present in a sample. It is possible to rapidly and easily detect the presence of phenylalanine and/or tyrosine in a sample by using the electrochemical aptamer biosensor of the present disclosure to effectively monitor information on tyrosine accumulation in the body of patients with phenylketonuria, thereby contributing to the treatment and management of patients with phenylketonuria.

### CROSS-REFERENCE TO RELATED APPLICATIONS

This application claims the priority of Korean Patent Application No. 10-2025-0015349 filed on Feb. 6, 2025, in the Korean Intellectual Property Office, the disclosure of which is incorporated herein by reference.

### REFERENCE TO SEQUENCE LISTING SUBMITTED ELECTRONICALLY

The content of the electronically submitted sequence listing (Name: P240131-Sequence listing.xml; Size: 2,790 bytes; and Date of Creation: Jan. 27, 2025) is herein incorporated by reference in its entirety.

### BACKGROUND

### Field

The present disclosure relates to an electrochemical aptamer biosensor capable of rapidly and easily detecting phenylalanine and/or tyrosine present in a sample.

### Description of the Related Art

Phenylketonuria (PKU) is a disease in which phenylalanine (Ph) is not properly metabolized but accumulates in the body due to a lack of phenylalanine hydroxylase (PAH).

Since phenylalanine, an essential amino acid, is found in a large quantity in protein-containing foods, PKU patients need to restrict their diet to prevent phenylalanine from accumulating. However, when these dietary restrictions are not strictly followed, symptoms such as intellectual disability, light brown hair, and lack of skin pigmentation may appear due to the accumulation of phenylalanine and its metabolites in the body. This disease mainly occurs in infancy and is known to be an autosomal recessive genetic disease caused by mutations in a PAH gene located on the long arm (12q22-q24.2) of chromosome 12.

PKU patients require life-long uninterrupted management and treatment to maintain a blood phenylalanine concentration within a normal range (2 to 6 mg/dL). To this end, it is very important to constantly check the level of tyrosine (Tyr) accumulation in the body and appropriately control the blood phenylalanine concentration.

A conventional method for measuring the concentrations of phenylalanine and tyrosine has been used for separation measurement using conventional chromatography methods. However, this method requires expensive analytical equipment and professionally trained analysts, and a process of preparing blood samples in an analyzable form is very complicated, so that the method has been a great burden for PKU patients who need to regularly measure tyrosine concentrations.

Therefore, there is a need to develop a rapid and sensitive method for measuring the concentrations of tyrosine and phenylalanine in the body while replacing expensive chromatography equipment.

### SUMMARY

Accordingly, while studying a new method capable of replacing a conventional chromatographic analysis method, the present inventors confirmed that an electrochemical aptamer biosensor manufactured by including a phenylalanine-specific binding aptamer and a polynucleotide sequence partially complementary thereto may rapidly distinguish and detect phenylalanine and/or tyrosine present in a sample without mutual signal interference, and then completed the present disclosure.

To achieve the object, there is provided an electrochemical aptamer biosensor for detecting phenylalanine or tyrosine, including: a reduced graphene oxide substrate; a phenylalanine-specific binding aptamer immobilized to the substrate; and a complementary polynucleotide having a sequence partially complementary to the aptamer and tagged with an electrochemical labeling material at a terminal.

Further, the present disclosure provides a method for manufacturing an electrochemical aptamer biosensor for detecting phenylalanine or tyrosine, including: 1) immobilizing a phenylalanine-specific binding aptamer onto the surface of a reduced graphene oxide electrode; and 2) hybridizing the immobilized aptamer with a complementary polynucleotide including a sequence partially complementary to the aptamer and tagged with an electrochemical labeling material at a terminal thereof.

Further, the present disclosure provides a method for manufacturing an electrochemical aptamer biosensor for detecting phenylalanine or tyrosine, including: 1) hybridizing a phenylalanine-specific binding aptamer with a complementary polynucleotide including a sequence partially complementary to the aptamer and tagged with an electrochemical labeling material at a terminal; and 2) coating the hybrid material of step 1 above on the surface of a reduced graphene oxide electrode.

Further, the present disclosure provides a method for detecting phenylalanine or tyrosine in a sample, including: providing a sample to be detected to an electrochemical aptamer biosensor of the present disclosure; and measuring changes in electrochemical signals according to the administration of the sample.

According to the present disclosure, it is possible to rapidly and easily detect the presence of phenylalanine and/or tyrosine in a sample by using the electrochemical aptamer biosensor of the present disclosure to effectively monitor information on tyrosine accumulation in the body of patients with phenylketonuria, thereby contributing to the treatment and management of patients with phenylketonuria.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a diagram showing a result of electrochemical reduction of graphene oxide and a result of confirming a surface structure using scanning electron microscopy (SEM);

FIG. 2 is a schematic diagram illustrating pc-DNA/MB design conditions of the present disclosure and selective binding of a PA-DNA aptamer according to the conditions;

FIG. 3 is a schematic diagram of a composite nanostructure electrode of metal nanoparticles and reduced graphene oxide;

FIG. 4 is a schematic diagram of measurement of tyrosine and phenylalanine using a composite nanostructure electrode of the present disclosure; and

FIG. 5 is a diagram illustrating results of confirming changes in electrochemical signals according to the presence of phenylalanine and tyrosine.

### DETAILED DESCRIPTION OF THE PREFERRED EMBODIMENT

The present disclosure relates to an electrochemical aptamer biosensor for detecting phenylalanine or tyrosine, including: a reduced graphene oxide substrate; a phenylalanine-specific binding aptamer immobilized to the substrate; and a complementary polynucleotide including a sequence partially complementary to the aptamer and tagged with an electrochemical labeling material at a terminal.

The electrochemical aptamer biosensor for detecting phenylalanine or tyrosine of the present disclosure may rapidly and easily detect the presence of phenylalanine and/or tyrosine in a sample.

In the present disclosure, the reduced graphene oxide substrate may be disposed on an electrode. The electrode that may be used in the present disclosure may be at least one selected from the group consisting of carbon (C), gold (Au), platinum (pt), and graphite.

In the present disclosure, a composite nanostructure electrode may be manufactured using metal nanoparticles and reduced graphene oxide. The metal nanoparticles may be included in a graphene oxide layer, and may include, for example, at least one selected from the group consisting of gold (Au), platinum (Pt), and silver (Ag), and are not limited to specific particles, but preferably, gold (Au) nanoparticles having excellent biocompatibility and electrical conductivity, ease of manufacture, and excellent chemical bonding.

The present disclosure relates to an electrochemical aptamer biosensor including g a working electrode, a reference electrode, and a counter electrode, and the working electrode may include at least one selected from the group consisting of gold (Au); silver (Ag); copper (Cu); platinum (Pt); titanium (Ti); nickel (Ni); tin (Sn); molybdenum (Mo); palladium (Pd); cobalt (Co); and alloys thereof; pyrolytic graphite; glassy carbon; carbon paste; perfluorocarbon (PFC); and carbon nanotubes (CNT), and may use a glassy carbon electrode polished with preferably glassy carbon, and more preferably alumina powder. The reference electrode may include at least one selected from the group consisting of a silver-silver chloride (Ag/AgCl) electrode, a calomel electrode, a mercury-mercury sulfate electrode, and a mercury-oxide mercury electrode, and preferably a silver-silver chloride (Ag/AgCl) electrode.

The phenylalanine-specific binding aptamer of the present disclosure is immobilized to the reduced graphene oxide substrate, and in the case of using other functional groups, the phenylalanine-specific binding aptamer may also be immobilized to metal nanoparticles included in the reduced graphene oxide substrate. In one embodiment of the present disclosure, an azide functional group (—N₃) was introduced to one terminal of the phenylalanine-specific binding aptamer, and radical ions were induced in the azide functional group by UV irradiation and then covalently linked to the reduced graphene oxide substrate.

In the present disclosure, the phenylalanine-specific binding aptamer may be a DNA or RNA sequence that may specifically bind to phenylalanine when the phenylalanine is present in a sample. When the phenylalanine is not present in the sample, the phenylalanine-specific binding aptamer is immobilized onto the substrate while hybridizing with the complementary polynucleotide including the sequence partially complementary thereto and tagged with the electrochemical labeling material at the terminal. However, when the phenylalanine is present in the sample, the phenylalanine-specific binding aptamer strongly binds to the phenylalanine while dehybridizing from the partially complementary sequence and causes changes in electrochemical signals.

The phenylalanine-specific binding aptamer of the present disclosure may be a polynucleotide having a length of 35 to 50 mer, preferably a length of 35 to 40 mer, and in a preferred embodiment of the present disclosure, an aptamer having a length of 38 mer was used. The specific phenylalanine-specific binding aptamer used in the present disclosure is represented by SEQ ID NO: 1, and therefore, the present disclosure provides an electrochemical aptamer biosensor, in which the phenylalanine-specific binding aptamer includes a polynucleotide sequence represented by SEQ ID NO: 1. Meanwhile, as long as the purpose described above may be achieved, a sequence having sequence homology of 80% or more, 90% or more, 95% or more, or 99% or more with the polynucleotide sequence represented by SEQ ID NO: 1 may be used.

The complementary polynucleotide including the sequence partially complementary to the phenylalanine-specific binding aptamer of the present disclosure and tagged with the electrochemical labeling material at the terminal may solve problems that the intensity of the electrochemical signal due to the oxidation/reduction of phenylalanine itself is small and thus difficult to be measured, and that the selectivity of the experiment may be lowered because the electrochemical oxidation/reduction reaction of phenylalanine is measured in a voltage range similar to the electrochemical oxidation/reduction of tyrosine.

More specifically, the complementary polynucleotide of the present disclosure is tagged with the electrochemical labeling material, and detects chemical signals thereof to indirectly detect phenylalanine. The signals emitted from the electrochemical labeling material are not only a strong electrochemical signal, but also does not interfere with a signal confirmed in the presence of tyrosine.

The electrochemical labeling material refers to a material that may generate an electrochemical signal according to a detection reaction. The electrochemical labeling material is required to have a small molecular size and high reactivity with DNA to ensure the sensitivity of the electrochemical signals. The electrochemical labeling material used in the method of the present disclosure may include methylene blue, hydroxyquinone, or ferrocene, and preferably, may be methylene blue.

Meanwhile, in the present disclosure, in order to increase the detection selectivity for tyrosine and phenylalanine, the lengths of the phenylalanine-specific binding aptamer and the partially complementary sequence were adjusted, thereby controlling their Gibbs free energy (G₀) range. Therefore, a G₀ value between the complementary polynucleotide of the present disclosure and the phenylalanine-specific binding aptamer may have a value between a G₀ value between tyrosine and the phenylalanine-specific binding aptamer, and a G₀ value between phenylalanine and the phenylalanine-specific binding aptamer, and their lengths may be 9 to 15 mer, preferably 10 to 12 mer. That is, the complementary polynucleotide is separated from the phenylalanine-specific binding aptamer when the phenylalanine is present in the sample, thereby inducing changes in the electrochemical signals.

In a preferred embodiment of the present disclosure, an aptamer represented by SEQ ID NO: 1 was used as the phenylalanine-specific binding aptamer, and a polynucleotide represented by SEQ ID NO: 2 labeled with methylene blue was used as the partially complementary sequence thereto. However, all partially complementary sequences that are partially complementary to the aptamer represented by SEQ ID NO: 1 and satisfy the G₀ condition may be included in the scope of the present disclosure.

The electrochemical aptamer biosensor of the present disclosure may separately detect phenylalanine or tyrosine in a sample, and simultaneously detect phenylalanine and tyrosine in the sample because the signals do not overlap or interfere with each other.

Further, the present disclosure provides a method for manufacturing an electrochemical aptamer biosensor for detecting phenylalanine or tyrosine (manufacturing method 1), including: 1) immobilizing a phenylalanine-specific binding aptamer on a reduced graphene oxide electrode surface; and 2) hybridizing the immobilized aptamer with a complementary polynucleotide including a sequence partially complementary to the aptamer and tagged with an electrochemical labeling material at a terminal thereof. Further, the present disclosure provides a method for manufacturing an electrochemical aptamer biosensor for detecting phenylalanine or tyrosine (manufacturing method 2), including: 1) hybridizing a phenylalanine-specific binding aptamer with a complementary polynucleotide including a sequence partially complementary to the aptamer and tagged with an electrochemical labeling material at a terminal; and 2) coating the hybrid material of step 1 above on the surface of a reduced graphene oxide electrode.

According to the manufacturing method 1, the phenylalanine-specific binding aptamer may be more efficiently immobilized on the surface of the reduced graphene oxide electrode, and according to the manufacturing method 2, the phenylalanine-specific binding aptamer and the polynucleotide partially complementary thereto may be hybridized more reliably. Therefore, in the manufacturing of the electrochemical aptamer biosensor for detecting phenylalanine or tyrosine of the present disclosure, either the manufacturing method 1 or the manufacturing method 2 may be appropriately selected and used.

In the manufacturing of the electrochemical aptamer biosensor for detecting phenylalanine or tyrosine of the present disclosure, when the manufacturing method 1 is selected, the phenylalanine-specific binding aptamer may first be immobilized on the surface of the reduced graphene oxide electrode by the following method. For example, the immobilizing step may be performed by the following method, but is not limited thereto. An azide functional group (—N₃) is immobilized to the 5′ end of a ‘PA-DNA aptamer’ represented by SEQ ID NO: 1, and a solution containing the ‘PA-DNA aptamer’ is positioned on the reduced graphene oxide electrode and then irradiated with a UV lamp for 5 minutes. The UV irradiation is performed by using a UV lamp with a wavelength of 365 nm, and fixing a distance between the electrode and the lamp to about 1 to 2 cm. The aptamer solution is treated in an amount of 5 to 10 μL, which is enough not to be dried for 5 minutes in an experimental environment while covering the surface area of the electrode at a concentration of 10 μM. Radical ions are generated in the azide functional group by UV irradiation, so that the PA-DNA aptamer covalently binds to the reduced graphene oxide. The description of the manufacturing method may be equally applied to the description of the electrochemical aptamer biosensor described above.

In addition, the present disclosure provides a method for detecting phenylalanine or tyrosine in a sample, including: providing a sample to be detected to an electrochemical aptamer biosensor; and measuring changes in electrochemical signals according to the administration of the sample.

The detection signal for the phenylalanine may be measured at −0.6 V to 0 V with respect to a reference electrode, and the detection signal for the tyrosine may be measured at 0.4 to 0.8 V with respect to the reference electrode.

In addition, since the changes in the electrochemical signals of the present disclosure may change in intensity depending on the concentration of the material to be detected in the sample, it is possible to simultaneously provide information on not only the presence or absence of the material to be detected in the sample, but also the concentration thereof.

Duplicated contents are omitted in consideration of the complexity of the present specification, and terms not defined otherwise in the present specification have the meanings commonly used in the art to which the present disclosure pertains.

Hereinafter, the present disclosure will be described in detail by Examples. However, the following Examples are just illustrative of the present disclosure, and the contents of the present disclosure are not limited to the following Examples.

### Example 1. Method for Manufacturing Composite Nanostructure Electrode

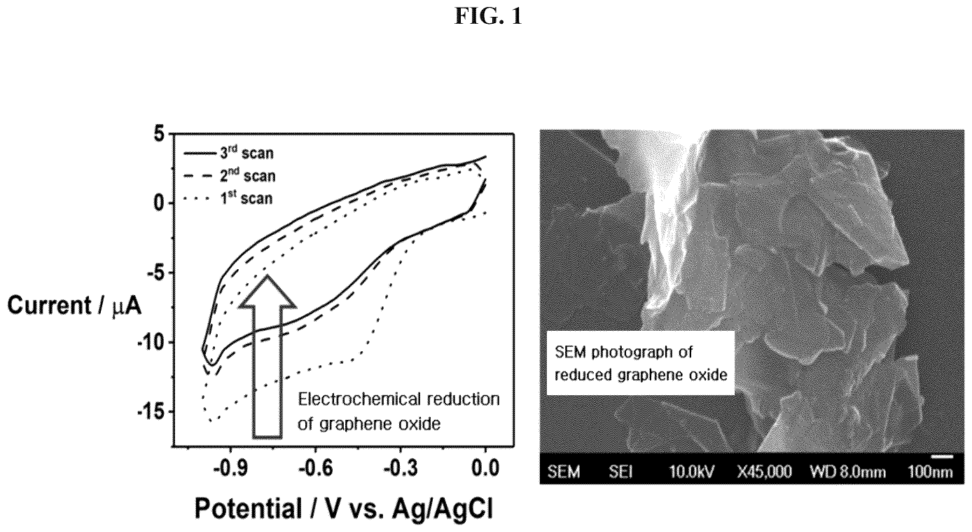

A composite nanostructure electrode using metal nanoparticles and reduced graphene oxide was manufactured by the following method. First, a graphene oxide solution was prepared by dispersing 0.003 g of graphene oxide (GO) in a 10 mM phosphate buffered saline (PBS) to prepare a graphene oxide solution with a concentration of 0.3 mg/mL. The prepared graphene oxide solution was sonicated for 1 hour at a power output of 150 watt/h using a probe with a diameter of 3 mm. A glassy carbon electrode polished with alumina powder of sizes 1.0 μm, 0.3 μm and 0.05 μm was used as a working electrode, Ag/AgCl was used as a reference electrode and a platinum wire was used as a counter electrode, and the three electrodes were immersed in the sonicated graphene oxide solution, and graphene oxide was electrochemically reduced using cyclic voltammetry (CV) by setting a scan rate of 0.1 V/s, a voltage range of −1 V to 0 V (vs. Ag/AgCl), and a cycle number of 3. The working electrode, the reference electrode, and the counter electrode converted to Electrochemically Reduced Graphene Oxide (ErGO) were washed with distilled water to remove a residual solution and impurities, thereby manufacturing a composite nanostructure electrode. FIG. 1 illustrated results of electrochemical reduction of graphene oxide and results of confirming a surface structure using scanning electron microscopy (SEM).

### Example 2. Method for Measuring Tyrosine Detection

20 μL of a PBS buffer solution was placed on the surface of the ErGO (Electrochemically Reduced Graphene Oxide) electrode manufactured in Example 1, and then an electrochemical system including a reference electrode and a counter electrode was set up for measurement. A voltage range from 0.4 V to 0.8 V (vs. Ag/AgCl) was measured by using differential pulse voltammetry (DPV) in the PBS solution to confirm the initial state of the electrode and check a background signal. Thereafter, each 4 μL of a real sample to be detected was injected into the PBS solution on the ErGO electrode, and a voltage range from 0.4 V to 0.8 V (vs. Ag/AgCl) was measured using DPS. The measurement was repeated a total of three times to measure signal changes according to the addition of a sample, and tyrosine detection is possible through this method.

### Example 3. Manufacture of Aptamer Electrode for Phenylalanine-Specific Detection

### 3.1 Binding of Phenylalanine-Specific DNA Aptamer (PA-DNA Aptamer)

An aptamer was designed to specifically detect phenylalanine present in a sample, and an aptamer electrode consisting of the aptamer was manufactured. The used phenylalanine-specific aptamer sequence was as follows.

Phenylalanine (PA)-DNA aptamer: 5′-CGACG AGGCT GGATG CATTC GCCGG ATGTT CGATG TCG-3′ (38 mer, SEQ ID NO: 1)

In order to immobilize the PA-DNA aptamer on the surface of the reduced graphene oxide electrode manufactured in Example 1, an azide functional group (—N₃) was added to the 5′-terminal of the ‘PA-DNA aptamer’ represented by SEQ ID NO: 1. Thereafter, the solution containing the PA-DNA aptamer was positioned on the reduced graphene oxide electrode and immobilized by irradiating a UV lamp.

As such, the step of immobilizing the PA-DNA aptamer on the surface of the reduced graphene oxide electrode may be performed by immobilizing the PA-DNA aptamer alone before the ‘step of hybridizing pc-DNA/MB’ to be described below; or performed by first hybridizing the PA-DNA aptamer with pc-DNA/MB and then immobilizing the hybridized material hybridized with pc-DNA/MB. The following experiment was performed by immobilizing the hybridized material hybridized with pc-DNA/MB.

### 3.2 Preparation and Hybridization of Pc-DNA/MB Complementary to PA-DNA Aptamer

### 3.2.1 Design of Pc-DNA/MB Sequence

The electrochemical signal caused by oxidation/reduction of phenylalanine itself was difficult to be measured due to its small intensity, and the electrochemical oxidation/reduction reaction of phenylalanine was measured in a similar voltage range to the electrochemical oxidation/reduction reaction of tyrosine, which may result in low selectivity of the experiment. To complement the problem, a sensor was designed to indirectly detect phenylalanine by detecting the electrochemical signal of methylene blue. For the purpose, a DNA sequence partially complementary to the PA-DNA aptamer was designed and tagged with methylene blue, in which the electrochemical oxidation/reduction reaction occurred well. The partially complementary DNA sequence designed in the present disclosure is referred to as ‘pc-DNA/MB’, which amplifies the signal of the aptamer electrode for phenylalanine and improves selectivity.

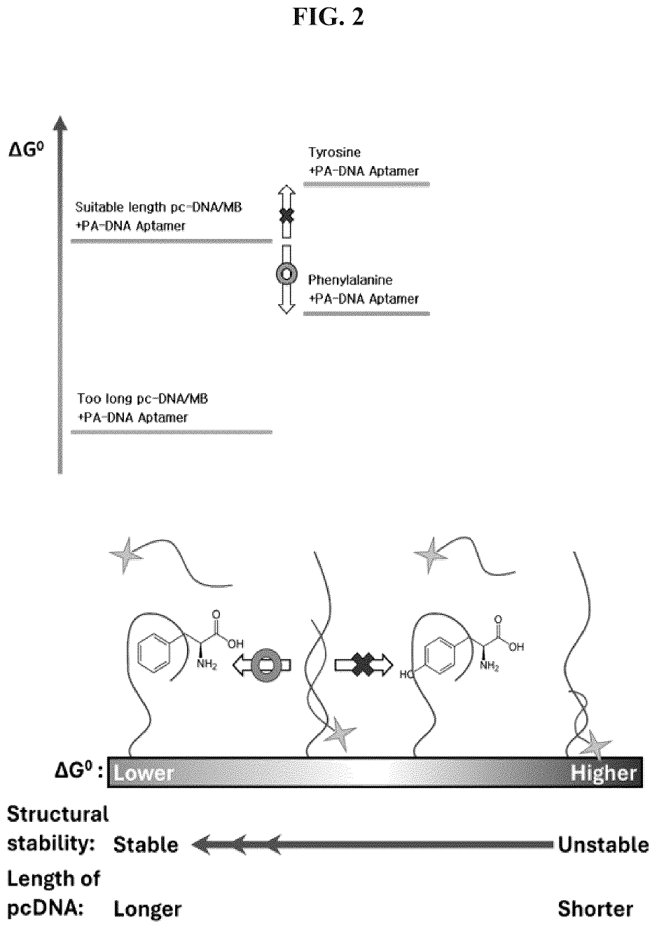

The pc-DNA/MB was designed to satisfy the following purposes. First, the pc-DNA/MB should not show signal changes for tyrosine, which had high structural similarity to phenylalanine. Selectivity for phenylalanine and tyrosine may be achieved by controlling the sequence length of pc-DNA/MB. A complementary sequence was designed by adjusting the length of pc-DNA/MB so that the G₀ values of pc-DNA/MB and PA-DNA are between a ΔG₀ value of the ‘tyrosine+PA-DNA aptamer’ structure and a ΔG₀ value of the ‘phenylalanine+PA-DNA aptamer’ structure. The principle in which the PA-DNA aptamer selectively bound to phenylalanine was illustrated in FIG. 2.

A partially complementary DNA sequence designed in the present disclosure was as follows.
pc-DNA/MB: 5′-Amino-AGC CTC GTC G-3′ (10-mer, SEQ ID NO: 2)

### 3.2.2 Hybridization of PA-DNA Aptamer and Pc-DNA/MB

20 μL of a ‘PA-DNA aptamer-N3’ solution (10 mM Tris-HCl, pH 7.4 solvent) diluted to a concentration of 10 μM and 20 μL of a ‘pc-DNA/MB’ solution (10 mM Tris-HCl, pH 7.4 solvent) diluted to a concentration of 10 μM were boiled at 95° C. for 5 minutes. Thereafter, the two solutions were mixed and boiled at 95° C. to induce hybridization for 10 minutes. After 10 minutes, the solution was removed from a double boiler and cooled to room temperature to achieve hybridization of the PA-DNA aptamer and pc-DNA/MB (PA-DNA aptamer+pc-DNA/MB′).

### 3.2.3 Manufacture of Electrochemical Aptamer Biosensor

5 μL of ‘PA-DNA aptamer+pc-DNA/MB’ at a 10 μM concentration was positioned on the reduced graphene oxide electrode manufactured by the electrochemical method, and then a UV lamp (wavelength: 325 nm) was positioned at 2 to 3 cm away from the electrode surface and irradiated UV for 5 minutes. After UV irradiation, the electrode was washed with tertiary distilled water for 3 minutes and dried with high-purity nitrogen (N2 gas, 99.999%). An electrochemical cell was constructed using the electrode manufactured through the method as a working electrode, a Pt wire as a counter electrode, Ag/AgCl as a reference electrode, and a 10 mM Tris-HCl pH 7.4 solution as a supporting electrolyte, and then used to measure phenylalanine/tyrosine.

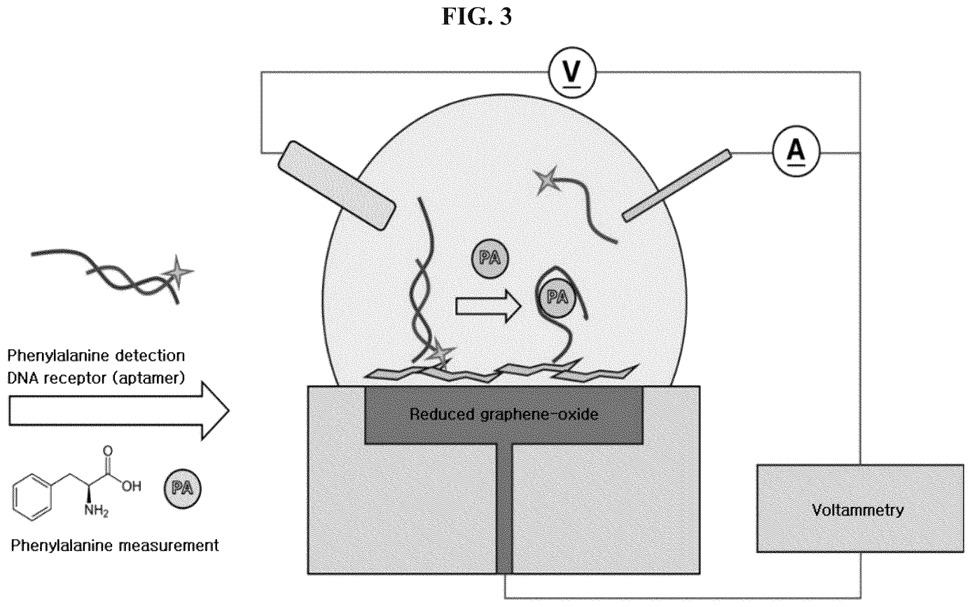

A schematic diagram of a composite nanostructure electrode of metal nanoparticles and reduced graphene oxide according to the present disclosure was illustrated in FIG. 3.

As illustrated in FIG. 3, while the PA-DNA aptamer and the pc-DNA/MB structure are combined with each other, methylene blue (MB) is positioned on the electrode surface, and a large electrochemical signal was measured. However, when phenylalanine is present in the sample and binds to the PA-DNA aptamer, the pc-DNA/MB is dehybridized by a competitive reaction, separated into single strands, and moves away from the electrode, thereby reducing the electrochemical signal. Since phenylalanine exists far from the electrode while binding to the PA-DNA aptamer, the electrochemical signal of phenylalanine is not generated even at a positive voltage, and thus the electrochemical signal of tyrosine may be measured without interference.

### Example 4. Confirmation of Detection Capacity of Tyrosine and Phenylalanine

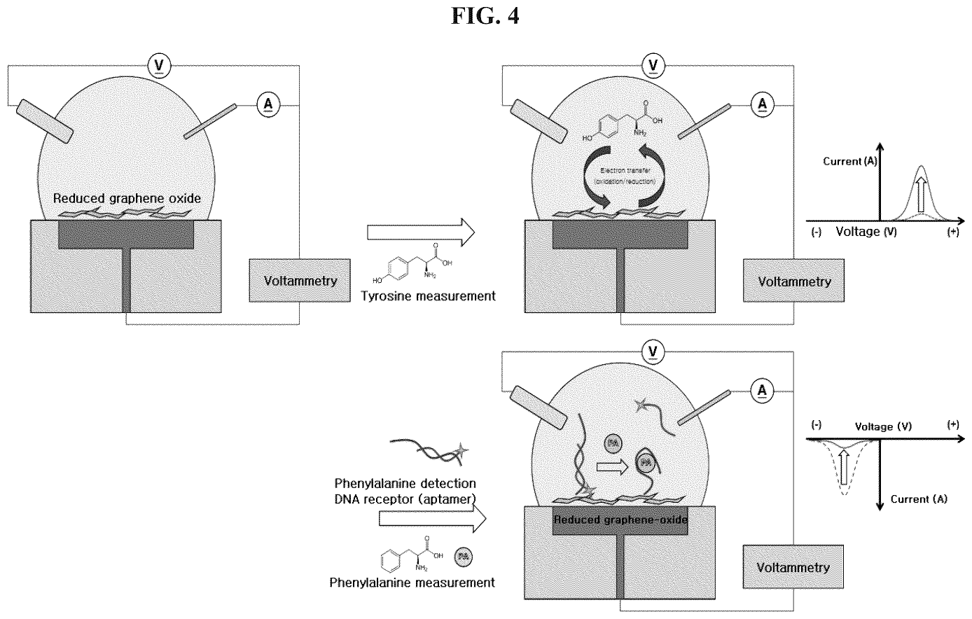

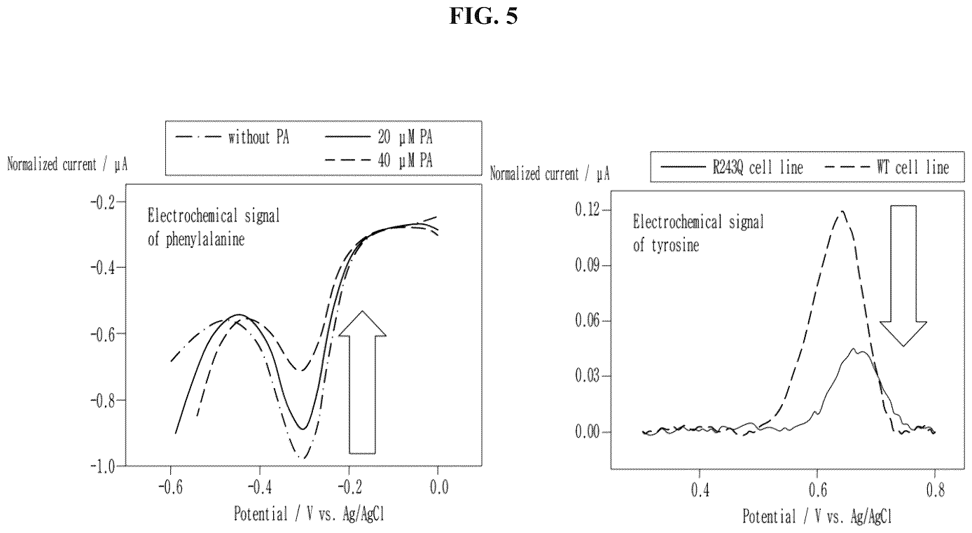

A schematic diagram of measuring tyrosine and phenylalanine using the manufactured composite nanostructure electrode of the present disclosure was illustrated in FIG. 4, and signal changes and detection effects according to the presence of phenylalanine and tyrosine in the detection sample were illustrated in FIG. 5. To verify the detection effects, R243Q cells expressing a PKU disease were used, and a normal cell line was used as a control group. A normal control group without expressing the PKU disease is cells in which the conversion reaction from phenylalanine to tyrosine proceeds smoothly, and thus the concentration of tyrosine is relatively higher.

As illustrated in FIGS. 4 and 5, the composite nanostructure electrode of the present disclosure may rapidly detect the presence of tyrosine in a sample by measuring changes in electric signals through an electron transfer (oxidation/reduction) reaction between reduced graphene oxide and tyrosine.

In addition, when phenylalanine is present in the sample, a PA-DNA+pc-DNA/MB structure immobilized on the reduced graphene oxide is dehybridized and the signal by methylene blue decreases, thereby rapidly detecting the presence of phenylalanine. In particular, when the PA-DNA aptamer does not binds to tyrosine, but phenylalanine binds to the PA-DNA aptamer by the PA-DNA+pc-DNA/MB structure of the present disclosure, phenylalanine exists far from the electrode, and thus an electrochemical signal of phenylalanine is not generated at a positive voltage, thereby enabling more accurate detection without interference with the electrochemical signal of tyrosine.

As illustrated in FIG. 5, the signal of methylene blue, which is a phenylalanine detection signal, appears in the range of −0.6 V to 0 V (vs. Ag/AgCl), while the detection signal of tyrosine appears in the range of 0.4 to 0.8 V (vs. Ag/AgCl) when measured by DPV. Thus, tyrosine and phenylalanine are selectively detected without a problem of overlapping electrochemical signals and concentrations thereof may be measured. In particular, the tyrosine concentration was measured to be lower in the R243Q cell line, a cell line expressing a PKU disease, than a normal control group, and thus it was confirmed that the electrochemical aptamer biosensor of the present disclosure may effectively transmit information on the relative concentration of tyrosine in the cell line.

As described above, specific parts of the present disclosure have been described in detail, and it will be apparent to those skilled in the art that these specific techniques are merely preferred embodiments, and the scope of the present disclosure is not limited thereto. Therefore, the substantial scope of the present disclosure will be defined by the appended claims and their equivalents.

### [Sequence List]

Sequence list electronic file attached.

What is claimed is:

1. An electrochemical aptamer biosensor for detecting phenylalanine or tyrosine, comprising:
a reduced graphene oxide substrate;
a phenylalanine-specific binding aptamer immobilized to the substrate; and
a complementary polynucleotide including a sequence partially complementary to the aptamer and tagged with an electrochemical labeling material at a terminal.

2. The electrochemical aptamer biosensor of claim 1, wherein the reduced graphene oxide substrate is disposed on an electrode.

3. The electrochemical aptamer biosensor of claim 1, wherein the phenylalanine-specific binding aptamer has a length of 35 to 50 mer.

4. The electrochemical aptamer biosensor of claim 1, wherein the phenylalanine-specific binding aptamer comprises a polynucleotide represented by SEQ ID NO: 1.

5. The electrochemical aptamer biosensor of claim 1, wherein a G₀ value between the complementary polynucleotide and the phenylalanine-specific binding aptamer has a value between a G₀ value between tyrosine and the phenylalanine-specific binding aptamer and a G₀ value between phenylalanine and the phenylalanine-specific binding aptamer.

6. The electrochemical aptamer biosensor of claim 1, wherein the complementary polynucleotide has a length of 9 to 15 mer.

7. The electrochemical aptamer biosensor of claim 1, wherein the electrochemical labeling material is methylene blue.

8. The electrochemical aptamer biosensor of claim 1, wherein the complementary polynucleotide is separated from the phenylalanine-specific binding aptamer when phenylalanine is present in the sample.

9. The electrochemical aptamer biosensor of claim 1, wherein the electrochemical aptamer biosensor simultaneously detects phenylalanine and tyrosine.

10. A method for manufacturing an electrochemical aptamer biosensor for detecting phenylalanine or tyrosine, comprising:
step 1, immobilizing a phenylalanine-specific binding aptamer on the surface of a reduced graphene oxide electrode; and
step 2, hybridizing the immobilized aptamer with a complementary polynucleotide comprising a sequence partially complementary to the aptamer and tagged with an electrochemical labeling material at a terminal thereof.

11. A method for manufacturing an electrochemical aptamer biosensor for detecting phenylalanine or tyrosine, comprising:
step 1, hybridizing a phenylalanine-specific binding aptamer with a complementary polynucleotide comprising a sequence partially complementary to the aptamer and tagged with an electrochemical labeling material at a terminal thereof; and
step 2, coating the hybrid material of step 1 above on the surface of a reduced graphene oxide electrode.

12. A method for detecting phenylalanine or tyrosine in a sample, comprising: providing a sample to be detected to the electrochemical aptamer biosensor of claim 1; and measuring changes in electrochemical signals according to the administration of the sample.

13. The method for detecting phenylalanine or tyrosine in the sample of claim 12, wherein a detection signal for the phenylalanine is measured at −0.6 V to 0 V with respect to a reference electrode, and a detection signal for the tyrosine is measured at 0.4 to 0.8 V with respect to the reference electrode.


SEQUENCE LISTING (2 sequences)

SEQ ID NO: 1 - DNA, 38 bp, synthetic construct
  source 1..38: mol_type = other DNA; note = PA-DNA aptamer
cgacgaggctggatgcattcgccggatgttcgatgtcg

SEQ ID NO: 2 - DNA, 10 bp, synthetic construct
  source 1..10: mol_type = other DNA; note = pc-DNA/MB
agcctcgtcg